(19) United States
(12) Patent Application Publication
(10) Pub. No.: US 2023/0054713 A1
(43) Pub. Date: Feb. 23, 2023

(54) METHOD FOR DETERMINING CONTRIBUTION RATE OF POLLUTION LOAD IN WATER QUALITY ASSESSMENT SECTION OF ANNULAR RIVER NETWORK SYSTEM BASED ON WATER QUANTITY CONSTITUTE

(71) Applicant: HOHAI UNIVERSITY, Nanjing (CN)

(72) Inventors: Peng WANG, Nanjing (CN); Yixin MA, Nanjing (CN); Zulin HUA, Nanjing (CN)

(21) Appl. No.: 17/809,399

(22) Filed: Jun. 28, 2022

(30) Foreign Application Priority Data
Aug. 16, 2021 (CN) 202110939056.3

(51) Int. Cl. G06Q 50/26; G01N 33/18

(52) U.S. Cl. CPC G06Q 50/26; G01N 33/18

(57) ABSTRACT

A method for determining a contribution rate of pollution load in a water quality assessment section of an annular river network system based on water quantity constitute comprises the following steps: defining all water quantity components (rainfall, pollution discharge and water diversion) according to a water quantity source condition of a river network water system in a research region; calculating a water quantity ratio of each water quantity component in each water quality assessment section; collecting flow rate of point source pollution and corresponding pollutant discharge amount, calculating a water quantity weighted average concentration of all point source pollutants, and calculating a runoff rate and a pollution load of all land use types and an average concentration of non-point source pollutants by utilizing a hydrological model and a pollution load model; and calculating the contribution rate of pollution load in the water quality assessment section.

### CROSS REFERENCES

This application claims priority to Chinese Patent Application Ser. No. CN202110939056.3 filed on 16 Aug. 2021.

### TECHNICAL FIELD

The present invention belongs to the field of environmental management technologies, and more particularly, to a method for determining a contribution rate of pollution load in a water quality assessment section of an annular river network system based on water quantity constitute.

### BACKGROUND

A water quality assessment section refers to a sampling section arranged to assess and monitor effects of pollution sources on both sides of a river reach on water quality to control pollutant discharge. The setting of the section is centered on improving a water environment quality, meeting current environmental management requirements such as water pollution prevention target and task assessment of a drainage basin and ranking of urban water environment quality. The quantification of a contribution rate of amount of pollution load in each region (or pollution control unit) at the location of water quality assessment section is beneficial for identifying a key pollution production region and clarifying a direction and focus of pollution management, and is of great significance to put forward a more targeted pollution control measure and water environment management scheme.

The contribution rate of the pollution load is usually calculated by the following two methods. In the first method, pollutant discharge amount of pollution control units need to be set as 0 one by one, pollution loads in the water quality assessment sections in different calculation schemes are predicted by water quantity and water quality models, by comparing with a pollution load in a normal discharge scheme, a contribution rate of a pollution load of a certain pollution control unit is counted, which means that only the contribution rate of one pollution control unit can be obtained by one calculation, so that the method has the disadvantages of cumbersome condition setting, low calculation efficiency and long research cycle. In the second method, a product of a water quantity constitute in the water quality assessment section and a corresponding pollutant concentration is directly used as the contribution rate of the pollution load, a physical concept of the method is clear, contribution rates of all pollution control units in the water quality assessment sections can be obtained by one calculation, and for a region or drainage basin with more pollution control units, the calculation efficiency of the method is much higher than that of the first method, but the difficulty of the method lies in determining ratios of the water quantity components in the assessment section.

A water system of the drainage basin is usually network-shaped, so that this network-shaped water system structure is called a river network. According to morphological characteristics of the river network, the river network may be categorized into dendritic river network and annular river network, as shown in FIG. 2. In mountainous and hilly regions with large terrain elevation changes, an upstream river system of the drainage basin usually has a main stream and tributaries, wherein the tributaries are similar to branches and the main stream is similar to a trunk, so that the water system structure of the whole drainage basin is similar to a structure from the branches to the trunk, and this river system is called the dendritic river network. In plain regions, a water system of a river channel is crisscross, a water flow has no fixed direction, the water system is in an annular structure, this water system is called the annular river network, and a downstream water system of the drainage basin in plain regions usually shows characteristics of the annular river network. For the dendritic river network, the tributaries gradually converge to the main stream, and a water quantity in a downstream section of the river channel must be converged from upstream water flows. Therefore, water quantity constitute may be obtained by calculating ratios of flow rates of the tributaries to a flow rate of the main stream. However, for the annular river network, especially in regions having numerous water conservancy projects and affected by tides, a water flow of the river channel is affected by rainfall, tides, operation modes of sluice pumps, water supply, water utilization, water consumption and water drainage in regions and boundaries, leading to an uncertain flow direction of the water flow of the river channel, and complicated source, destination and movement characteristics of the water flow, and it is usually difficult to determine the water quantity constitute in the water quality assessment section.

### SUMMARY

Object of the invention: in order to overcome the defect in the prior art that it is difficult to determine a contribution rate of pollution load in a water quality assessment section of a water system of an annular river network due to complicated source, destination and movement characteristics of a water flow, the present invention provides a method for determining a contribution rate of pollution load in a water quality assessment section of an annular river network system based on water quantity constitute.

Technical solutions: a method for determining a contribution rate of pollution load in a water quality assessment section of an annular river network system based on water quantity constitute comprises the following steps of:
(1) determining water quantity components in a research region, comprising a plurality of rainfall runoffs, wastewater discharge and water diversion;
(2) constructing a river network water quantity constitute model, regarding the all water quantity components as conservative substances, and calculating water quantity ratios of the water quantity components in each water quality assessment section;
(3) collecting pollution loads and wastewater quantity of all wastewater discharges, and calculating a weighted average concentration of all wastewater discharge pollutants; using a hydrological model and a pollution load model to calculate pollution loads, and water yields of all rainfall runoffs, and calculate a weighted average concentration of rainfall runoff pollutants; and acquiring a weighted average concentration of water diversion pollutants; and
(4) according to the water quantity ratios of the water quantity components and the weighted average concentration of the pollutants, calculating contribution rates of the pollutants of the all water quantity components in the water quality assessment section in the research region.
(5) Further, in step (2), meteorological conditions of precipitation and evaporation and land use conditions in the research region are input into the hydrological model to calculate water yields of all land uses, and the water yields of the land use are taken as the water quantity components of the rainfall runoff; collected wastewater discharge rates are taken as the water quantity components of the wastewater discharge; and water diversion rates outside the research region are taken as the water quantity components of the water diversion; and
(6) the water quantity ratio of each water quantity component in the water quality assessment section is calculated by the water quantity constitute model, which is ϕₜ^{j}, and ϕₜ^{j} is a water quantity ratio of an i^{th} water quantity component in a j^{th} water quality assessment section.

Further, in step (1), the rainfall runoff is defined as a non-point source and the wastewater discharge is defined as a point source, the non-point source is classified into domestic pollution of rural residents, planting pollution, livestock and poultry pollution and urban surface runoff pollution; and the point source is classified into direct discharge industrial pollution, wastewater treatment plant pollution and other untreated domestic pollution.

Further, in step (3), the weighted average concentration of the non-point source pollutants is calculated according to a total load rate of various non-point source pollutants divided by a flow rate of wastewater and a runoff rate of all land uses; the weighted average concentration of the point source pollutants is calculated according to a total load of all point source pollutants divided by the corresponding flow rate of wastewater;

a calculation formula is:

$$\overline{{C}_{i}} = \frac{{\sum }_{i = 1}^{m} {WL}_{i}}{{\sum }_{i = 1}^{m} {W}_{i}} \times 100 \qquad ( 1 )$$

wherein C̅ᵢ̅ is a weighted average concentration of pollutants of an i^{th} water quantity component, mg/L; WLᵢ is a pollutant load rate of the i^{th} water quantity component, t/a, which is collected from data or calculated by the pollution load model; Wᵢ is a flow rate of the i^{th} water quantity component, 10,000 m³/a, which is collected from data or predicted by the hydrological model; and m is a number of pollution classifications of the non-point source and the point source, wherein 4 non-point sources and 3 point sources are provided.

Further, in step (3), the weighted average concentration of the water diversion pollutants is determined by water quality monitoring data.

Further, in step (4), a calculation method of the contribution rates of the pollutants of the water quantity components in the water quality assessment section in the research region is:

$${P}_{i}^{j} = \frac{{ϕ}_{i}^{j} \cdot \overline{{C}_{i}}}{{\sum }_{i = 1}^{n} {ϕ}_{i}^{j} \cdot \overline{{C}_{i}}} \qquad ( 2 )$$

wherein Pₜ^{j} is a contribution rate of load of the pollutants of the i^{th} water quantity component in the j^{th} water quality assessment section; ϕₜ^{j} is the water quantity ratio of the i^{th} water quantity component in the j^{th} water quality assessment section; C̅ᵢ̅ is the weighted average concentration of the pollutants of the i^{th} water quantity component, mg/L; and n is a number of the water quantity components.

Further, in step (2), a construction method of the river network water quantity constitute model comprises: based on a water quality model, regarding the water quantity components as the conservative substances, regardless of transformation and fate, and representing model results as ratios of each water quantity components, wherein n rivers are set, corresponding flow rates of rivers L₁, L₂, . . . , Lₙ₋₁ are q₁, q₂, . . . , qₙ₋₁ respectively, and the n−1 rivers all flow to the river Lₙ, which means that a water quantity of the river Lₙ is composed of water quantity of the rivers L₁, L₂, . . . , Lₙ₋₁, so that a flow rate of Lₙ is that q=q₁+q₂+, . . . , +qₙ₋₁, and ratios of the water quantity are L₁: q₁/q, L₂: q₂/q, . . . , Lₙ₋₁: qₙ₋₁/q respectively; and assuming that concentrations of the conservative substances entering the river with the water flow are all 1.0, concentrations of the conservative substances in the river Lₙ are L₁: q₁/q, L₂: q₂/q, . . . , Lₙ₋₁: qₙ₋₁/q respectively; and determining the water quantity ratios of the water quantity components according to the concentrations of the conservative substances in the river.

Beneficial effects: in the method for determining the contribution rate of pollution load in the water quality assessment section of the annular river network system based on water quantity constitute compared with the prior art, by defining all water quantity components such as rainfall, sewage discharge and water diversion, a water quantity constitute calculation problem is converted into a conservative substance concentration calculation problem, the water quantity ratios of the water quantity components in each water quality assessment section are calculated, the average concentration of each pollutants is counted by combining the hydrological model and the pollution load model at the same time, and results of the water quantity constitute and the average concentration of the pollutants are integrated to calculate the contribution rate of the pollutant load in the water quality assessment section, thus overcoming a difficult problem of calculating the contribution rate of the pollution load in a complicated annular river network region with an uncertain water flow direction, and having an application value for a pollution traceability research in regions with similar reciprocating flow hydrological characteristics.

### BRIEF DESCRIPTION OF THE DRAWINGS

FIG. 1A is a schematic diagram of a calculation principle of water quantity constitute with a river network number;

FIG. 1B is a schematic diagram of a calculation principle of water quantity constitute with a flow rate and a concentration;

FIG. 2 is a schematic structural diagram of a dendritic river network and an annular river network;

FIG. 3 is an administrative division map of a town level of an A city;

FIG. 4 is a distribution diagram of state controlled and provincial controlled sections of the A city;

FIG. 5 is a general graph of a river network water system of the A city;

FIG. 6 is a diagram of a contribution rate of pollution load of the A city.

### DETAILED DESCRIPTION

The present invention is further described hereinafter with reference to the drawings and the specific embodiments.

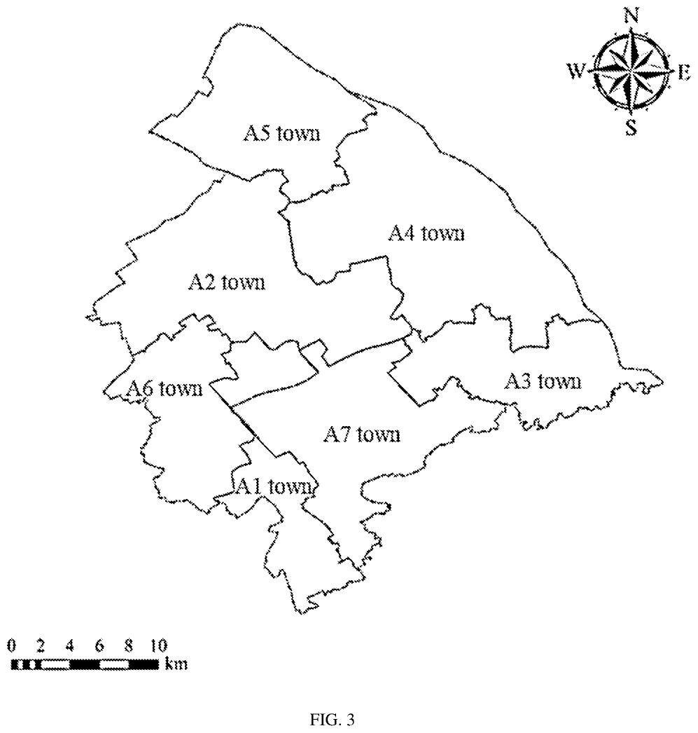

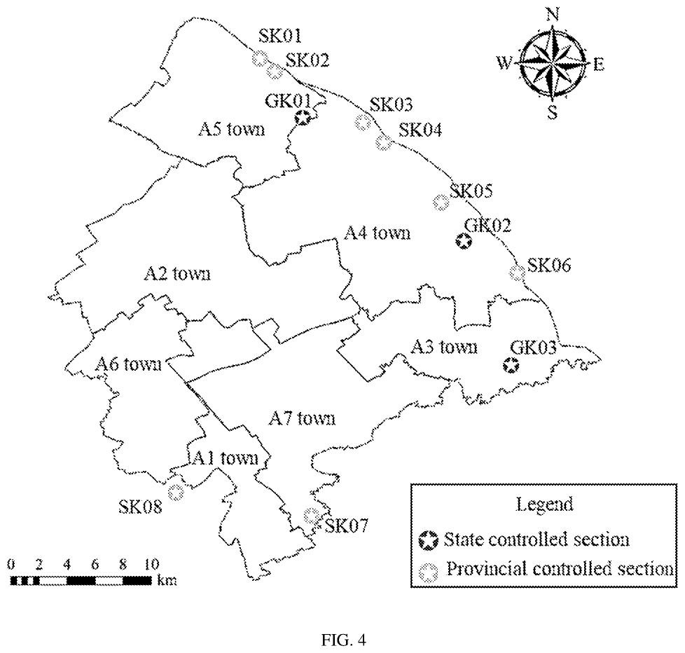

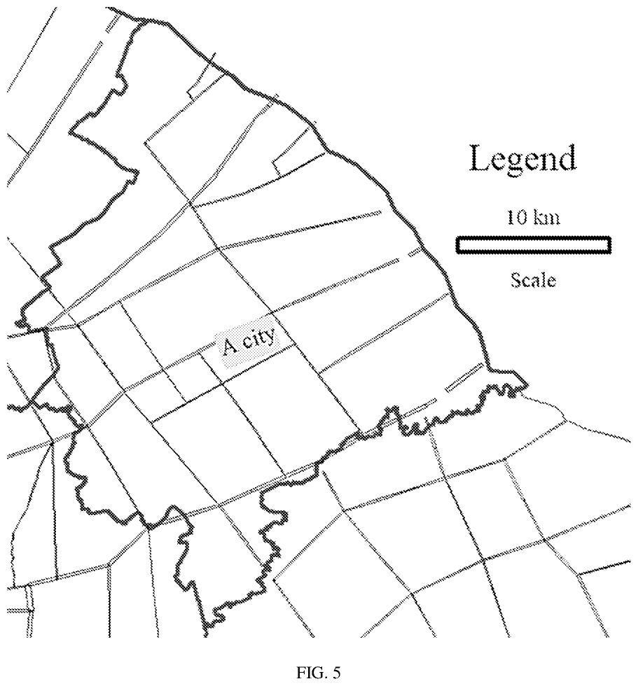

In the embodiment, a certain city (A city) in eastern China is taken as an example to analyze a contribution rate of pollution load in a water quality assessment section. FIG. 3 is an administrative division map of a town level of the A city. The region is low and flat, has many water conservancy projects, and is affected by tides, thus leading to an uncertain water flow direction of a river channel, and belonging to a typical annular river network. The city has 3 state controlled sections and 8 provincial controlled sections, and spatial distribution of the sections is shown in FIG. 4. Basic data of meteorology, hydrology, water system, water conservancy project, land use, pollution source and water quality monitoring of the A city are sorted out, analyzed and generalized, and a water quantity and water quality mathematical model of a river network in the region is constructed. A general graph of the water system is shown in FIG. 5.

A method for determining a contribution rate of pollution load in a water quality assessment section of an annular river network system based on water quantity constitute comprises the following steps.

(1) Determination of water quantity components of A city

Taking an administrative region as a pollution control unit, pollution discharges of 7 town-level administrative regions (A1 town to A7 town) in the A city are defined as 7 water quantity components respectively, and pollution discharges of a B city and a C city adjacent to the A city and other regions in a drainage basin are defined as 3 water quantity components respectively. The pollution discharges comprise both non-point source (rainfall runoff) and point source (wastewater discharge). The non-point source comprises four classifications of domestic pollution of rural residents, planting pollution, livestock and poultry pollution and urban surface runoff pollution; and the point source comprises three classifications of direct discharge industrial pollution, wastewater treatment plant pollution and other untreated domestic pollution. In addition, water diversion from outside in the region is set as one water quantity component.

(2) Calculation of water quantity ratios of water quantity components

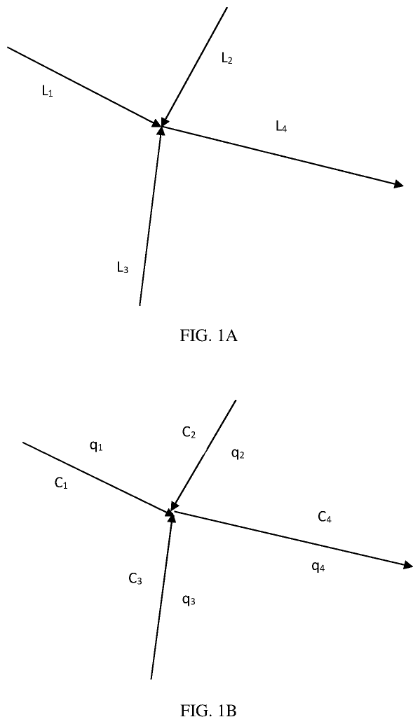

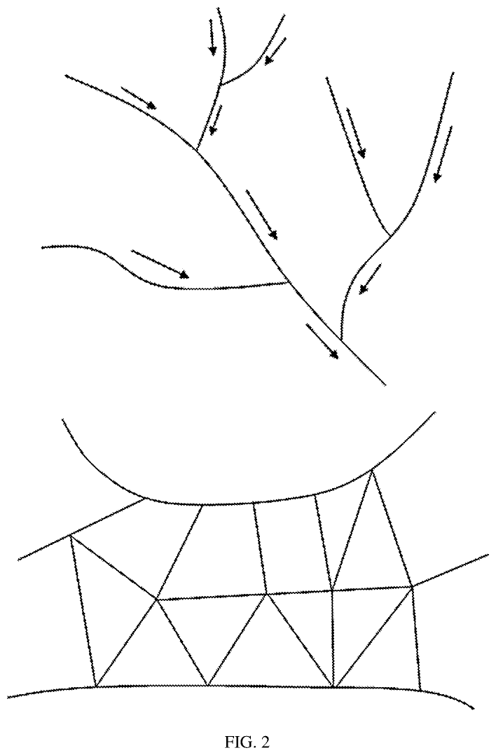

A river network water quantity constitute model is constructed, and based on a water quality model, the former regards the water quantity components as conservative substance, regardless of transformation and fate. In addition, model calculation results are represented as ratios of the water sources. If all water sources are considered, then a sum of the water quantity components of any model object is equal to 1.0. FIG. 1A-B is a schematic diagram of a basic calculation principle of water quantity constitute.

As shown in FIG. 1A, assuming that four rivers L₁, L₂, L₃ and L₄ are provided, corresponding flow rates of the rivers L₁, L₂ and L₃ are q₁, q₂ and q₃ respectively, and the flow rates of the three rivers all flow to the river L₄, which means that a water quantity of the river L₄ is composed of water quantity of the rivers L₁, L₂ and L₃, so that a flow rate of the river L₄ is q=q₁+q₂+q₃, and the water quantity constitute are L₁: q₁/q, L₂: q₂/q and L₃: q₃/q respectively.

As shown in FIG. 1B, assuming that a conservative substance C₁ enters the river L₁ along with a water flow, the substance is not degraded during the movement along with the water flow. Similarly, conservative substances C₂ and C₃ enter the rivers L₂ and L₃ along with the water flow, assuming that concentrations of conservative substances in rivers are all 1.0, the 3 conservative substances are fully mixed at a confluence and then enter the river L₄, and then concentrations of the conservative substances C₁, C₂ and C₃ in the river L₄ are q₁/q, q₂/q and q₃/q respectively. The concentrations of the conservative substances are exactly equal to ratios of water quantity carrying the substances. Therefore, as long as types of conservative substances in different water sources are defined, and the water quality model is used to calculate a change process of the concentrations of the conservative substances in the rivers with time, water quantity constitute of each river reach may be obtained.

According to the above definition of the water quantity component, assuming that sewage discharges and external water diversions of 7 town-level administrative regions in the A city, the B city, the C city and other regions contain 11 conservative substances with a concentration of 1.0 respectively, the concentrations of the 11 conservative substances in the water quality assessment section are calculated by the constructed river network water quantity constitute model according to the above calculation method of the water quantity constitute, which means that water quantity ratios of 11 water quantity components in the water quality assessment section are recorded as ϕₜ^{j}, and ϕₜ^{j} is a water quantity ratio of an i^{th} water quantity component in a j^{th} water quality assessment section.

(3) Calculation of weighted average concentration of water quantity

A weighted average concentration of non-point source pollutants is calculated according to a total load rate of all non-point source pollutants divided by a flow rate of wastewater and a runoff rate of all land uses, the flow rate of wastewater and the runoff rate of land uses are calculated by a hydrological model and a pollution load model. The hydrological model and the pollution load model are professional mathematical models for simulating runoff generation and confluence in the drainage basin/region and calculating the pollution load, with many types, which are selected according to characteristics of meteorology, hydrology, soil, topography and pollution sources in a research region. A weighted average concentration of point source pollutants is calculated according to a total load of all point source pollutants divided by a corresponding flow rate of wastewater, and the pollution load and the wastewater quantity of the wastewater discharge are obtained by collection. A weighted average concentration of water diversion pollutants is determined by water quality monitoring data.

A calculation formula of the weighted average concentration of the pollutants is:

$$\overline{{C}_{i}} = \frac{{\sum }_{i = 1}^{m} {WL}_{i}}{{\sum }_{i = 1}^{m} {W}_{i}} \times 100 \qquad ( 1 )$$

wherein C̅ᵢ̅ is a weighted average concentration of pollutants of an i^{th} water quantity component, mg/L; WLᵢ is a pollutant load rate of the i^{th} water quantity component, t/a, which is collected from data or calculated by the pollution load model; Wᵢ is a flow rate of the i^{th} water quantity component, 10,000 m³/a, which is collected from data or predicted by the hydrological model; and m is a number of pollution classifications of the non-point source and the point source, wherein there are 4 non-point sources of domestic pollution of rural residents, planting pollution, livestock and poultry pollution and urban surface runoff pollution and 3 point sources of direct discharge industrial pollution, wastewater treatment plant pollution and other untreated domestic pollution.

(4) Calculation of contribution rate of pollution load

According to the water volume ratios of the all water quantity components and the weighted average concentration of the pollutants, contribution rates of the pollutants of the all water quantity components in the water quality assessment section in the research region are calculated, and a calculation method is:

$${P}_{i}^{j} = \frac{{ϕ}_{i}^{j} \cdot \overline{{C}_{i}}}{{\sum }_{i = 1}^{n} {ϕ}_{i}^{j} \cdot \overline{{C}_{i}}} \qquad ( 2 )$$

wherein Pₜ^{j} is a contribution rate of load of the pollutants of the i^{th} water quantity component in the j^{th} water quality assessment section; ϕₜ^{j} is the water quantity ratio of the i^{th} water quantity component in the j^{th} water quality assessment section; C̅ᵢ̅ is the weighted average concentration of the pollutants of the i^{th} water quantity component, mg/L; and n is a number of the water quantity components.

Through experiment researches on all investigation sections in the A city, results are as follows.

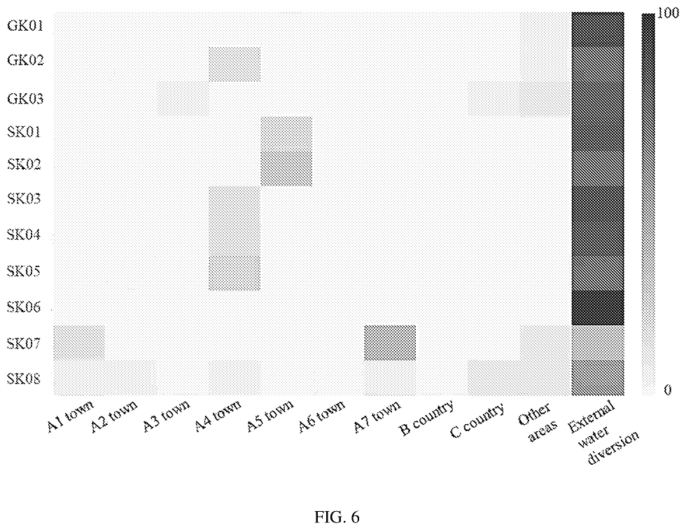

Taking total phosphorus as an example, according to formula (2), contribution rates of pollution loads of total phosphorus in 3 state controlled sections and 8 provincial controlled sections in 7 town-level administrative regions of the A city, the adjacent B city and C city, the other regions and the external water diversion are counted. Results are shown in Table 1 and FIG. 6.

TABLE 1
Contribution rates of loads of total phosphorus in water
quality assessment sections in administrative regions

| | | Contribution rate (%) | | | | | | | | | | |
|---|---|---|---|---|---|---|---|---|---|---|---|---|
| | | | | | | | | | | | | External |
| Level of | Name of | A1 | A2 | A3 | A4 | A5 | A6 | A7 | B | C | Other | water |
| section | section | town | town | town | town | town | town | town | city | city | regions | diversion |
| State | GK01 | 0.03 | 0.77 | 0.00 | 0.73 | 1.92 | 0.36 | 0.00 | 1.18 | 1.64 | 4.93 | 88.42 |
| controlled | GK02 | 0.25 | 2.47 | 2.87 | 15.27 | 0.01 | 0.39 | 0.12 | 0.46 | 2.41 | 3.17 | 72.58 |
| | GK03 | 0.70 | 0.36 | 4.33 | 0.30 | 0.00 | 0.50 | 1.56 | 0.37 | 4.88 | 8.88 | 78.12 |
| Provincial | SK01 | 0.00 | 0.60 | 0.00 | 0.13 | 18.12 | 0.00 | 0.00 | 0.01 | 0.00 | 0.00 | 81.14 |
| controlled | SK02 | 0.00 | 1.07 | 0.00 | 0.23 | 26.85 | 0.00 | 0.00 | 0.02 | 0.00 | 0.00 | 71.83 |
| | SK03 | 0.00 | 0.43 | 0.01 | 12.87 | 0.33 | 0.03 | 0.00 | 0.01 | 0.04 | 0.04 | 86.24 |
| | SK04 | 0.01 | 0.88 | 0.02 | 14.54 | 0.80 | 0.06 | 0.01 | 0.05 | 0.18 | 0.30 | 83.14 |
| | SK05 | 0.08 | 2.24 | 0.01 | 21.27 | 0.08 | 0.41 | 0.03 | 0.27 | 1.65 | 1.62 | 72.35 |
| | SK06 | 0.02 | 0.20 | 1.69 | 1.74 | 0.00 | 0.01 | 0.30 | 0.00 | 0.00 | 0.08 | 95.96 |
| | SK07 | 13.19 | 1.18 | 1.01 | 1.66 | 0.03 | 0.43 | 37.61 | 0.59 | 2.50 | 8.23 | 33.57 |
| | SK08 | 5.00 | 3.49 | 1.91 | 4.30 | 0.06 | 2.60 | 6.97 | 1.73 | 10.32 | 11.08 | 52.54 |

(5) According to the calculation in step (4), a list of contribution rates of pollution loads of total phosphorus of 11 water quantity components in the A city is obtained.

(6) According to the list of contribution rates of pollution loads obtained in step (5), the contribution rates of all water quantity components are ranked in each water quality assessment section. Taking the GK01 state controlled section as an example, the contribution rates of the 11 water quantity components are sequentially as follows: external water diversion (88.42%)>other regions (4.93%)>A5 town (1.92%)>C city (1.64%)>B city (1.18%)>A2 town (0.77%)>A4 town (0.73%)>A6 town (0.73%)>A1 town (0.03%)>A3 town and A7 town (0.00%). It is sequentially determined whether the weighted average concentrations of total phosphorus of all water quantity components exceed a pollutant discharge standard. If the weighted average concentrations exceed a standard threshold, sewage treatment device is constructed in the GK01 state controlled section in which the water quantity component are discharged to control total phosphorus discharge concentrations of the water quantity components; and if the weighted average concentrations do not exceed the standard threshold, then controlling discharge amount of the water quantity component; thus the contribution rates of the water quantity components in all assessment sections of the A city are controlled by this.

Those described above are merely the preferred embodiments of the present invention, and it should be pointed out that those of ordinary skills in the art may further make improvements and decorations without departing from the principle of the present invention, and these improvements and decorations should also be regarded as falling within the scope of protection of the present invention.

What is claimed is:

1. A method for determining a contribution rate of pollution load in a water quality assessment section of an annular river network system based on water quantity constitute, comprising the following steps of:
i) determining water quantity components in a research region, comprising a plurality of rainfall runoffs, wastewater discharge and water diversion;
ii) constructing a river network water quantity constitute model, regarding the all water quantity components as conservative substances, and calculating water quantity ratios of the water quantity components in each water quality assessment section, wherein a construction method of the river network water quantity constitute model comprises: based on a water quality model, regarding the all water quantity components as the conservative substances, regardless of transformation and fate, and representing model results as volume ratios of all water quantity components, wherein n rivers are set, corresponding flow rates of rivers L₁, L₂, . . . , Lₙ₋₁ are q₁, q₂, . . . , qₙ₋₁ respectively, and the n−1 rivers all flow to the river Lₙ, which means that a water quantity of the river Lₙ is composed of water quantity of the rivers L₁, L₂, . . . , Lₙ₋₁, so that a flow rate of Lₙ is that q=q₁+q₂+, . . . , +qₙ₋₁, and ratios of the water quantity are L₁: q₁/q, L₂: q₂/q, . . . Lₙ₋₁: qₙ₋₁/q respectively; and assuming that concentrations of the conservative substances entering the river with the water flow are all 1.0, concentrations of the all conservative substances in the river Lₙ are L₁: q₁/q, L₂: q₂/q, . . . , Lₙ₋₁: qₙ₋₁/q respectively; and determining the water quantity ratios of the all water quantity components according to the concentrations of the conservative substances in the river;
iii) collecting pollution loads and wastewater quantity of all wastewater discharges, and calculating a weighted average concentration of all wastewater discharge pollutants; using a hydrological model and a pollution load model to calculate a pollution load, a wastewater quantity and a water yield of land use types of all rainfall runoffs, and calculate a weighted average concentration of rainfall runoff pollutants; and acquiring a weighted average concentration of water diversion pollutants;
iv) according to the water quantity ratios of the all water quantity components and the weighted average concentration of the pollutants, calculating contribution rates of the pollutants of the all water quantity components in the water quality assessment section in the research region;
v) according to the calculation in step iv), obtaining a list of contribution rates of pollution loads of all water quantity components; and
vi) according to the list of contribution rates of pollution loads obtained in step v), ranking the contribution rates of all water quantity components in the water quality assessment section, sequentially determining whether the weighted average concentrations of pollutants exceed a pollutant discharge standard, and if the weighted average concentrations exceed a standard threshold, then constructing sewage treatment devices to control pollution discharge concentrations of the water quantity components; and if the weighted average concentrations do not exceed the standard threshold, then controlling discharge amounts of the water quantity components.

2. The method for determining the contribution rate of pollution load in the water quality assessment section of the annular river network system based on water quantity constitute according to claim 1, wherein in the step ii), meteorological conditions of precipitation and evaporation and land use conditions in the research region are input into the hydrological model to calculate runoff rates of all land use types, and the water yields of the land uses are taken as the water quantity components of the rainfall runoff; collected wastewater discharge quantity are taken as the water quantity components of the wastewater discharge; and water diversion quantity outside the research region are taken as the water quantity components of the water diversion; and
the water quantity ratio of each water quantity component in the water quality assessment section is calculated by the water quantity constitute model, which is ϕₜ^{j}, and ϕₜ^{j} is a water quantity ratio of an i^{th} water quantity component in a j^{th} water quality assessment section.

3. The method for determining the contribution rate of pollution load in the water quality assessment section of the annular river network system based on water quantity constitute according to claim 1, wherein in the step i), the rainfall runoff is defined as a non-point source and the wastewater discharge is defined as a point source, the non-point source is classified into domestic pollution of rural residents, planting pollution, livestock and poultry pollution and urban surface runoff pollution; and the point source is classified into direct discharge industrial pollution, wastewater treatment plant pollution and other untreated domestic pollution.

4. The method for determining the contribution rate of pollution load in the water quality assessment section of the annular river network system based on water quantity constitute according to claim 2, wherein in the step iii), the weighted average concentration of the non-point source pollutants is calculated according to a total load rate of non-point source pollutant divided by a flow rate of wastewater and a runoff rate of all land uses; the weighted average concentration of the point source pollutant is calculated according to a total load rate of all point source pollutant divided by the corresponding flow rate of wastewater;
a calculation formula is:
$$\overline{{C}_{i}} = \frac{{\sum }_{i = 1}^{m} {WL}_{i}}{{\sum }_{i = 1}^{m} {W}_{i}} \times 100 \qquad ( 1 )$$
wherein C̅ᵢ̅ is a weighted average concentration of pollutants of an i^{th} water quantity component, mg/L; WLᵢ is a pollutant load rate of the it water quantity component, t/a, which is collected from data or calculated by the pollution load model; Wᵢ is a flow rate of the i^{th} water quantity component, 10,000 m³/a, which is collected from data or predicted by the hydrological model; and m is a number of pollution classifications of the non-point source and the point source, wherein 4 non-point sources and 3 point sources are provided.

5. The method for determining the contribution rate of pollution load in the water quality assessment section of the annular river network system based on water quantity constitute according to claim 1, wherein in step iii), the weighted average concentration of the water diversion pollutants is determined by water quality monitoring data.

6. The method for determining the contribution rate of pollution load in the water quality assessment section of the annular river network system based on water quantity constitute according to claim 2, wherein in step iii), the weighted average concentration of the water diversion pollutants is determined by water quality monitoring data.

7. The method for determining the contribution rate of pollution load in the water quality assessment section of the annular river network system based on water quantity constitute according to claim 3, wherein in step iii), the weighted average concentration of the water diversion pollutants is determined by water quality monitoring data.

8. The method for determining the contribution rate of pollution load in the water quality assessment section of the annular river network system based on water quantity constitute according to claim 4, wherein in step iii), the weighted average concentration of the water diversion pollutants is determined by water quality monitoring data.

9. The method for determining the contribution rate of pollution load in the water quality assessment section of the annular river network system based on water quantity constitute according to claim 1, wherein in step iv), a calculation method of the contribution rates of the pollutants of the all water quantity components in the water quality assessment section in the research region is:
$${P}_{i}^{j} = \frac{{ϕ}_{i}^{j} \cdot \overline{{C}_{i}}}{{\sum }_{i = 1}^{n} {ϕ}_{i}^{j} \cdot \overline{{C}_{i}}} \qquad ( 2 )$$
wherein Pₜ^{j} is a contribution rate of load of the pollutants of the i^{th}, water quantity component in the j^{th} water quality assessment section; ϕₜ^{j} is the water quantity ratio of the i^{th} water quantity component in the j^{th} water quality assessment section; C̅ᵢ̅ is the weighted average concentration of the pollutants of the i^{th} water quantity component, mg/L; and n is a number of the water quantity components.

10. The method for determining the contribution rate of pollution load in the water quality assessment section of the annular river network system based on water quantity constitute according to claim 2, wherein in step iv), a calculation method of the contribution rates of the pollutants of the all water quantity components in the water quality assessment section in the research region is:
$${P}_{i}^{j} = \frac{{ϕ}_{i}^{j} \cdot \overline{{C}_{i}}}{{\sum }_{i = 1}^{n} {ϕ}_{i}^{j} \cdot \overline{{C}_{i}}} \qquad ( 2 )$$
wherein Pₜ^{j} is a contribution rate of load of the pollutants of the i^{th} water quantity component in the j^{th} water quality assessment section; ϕₜ^{j} is the water quantity ratio of the i^{th} water quantity component in the j^{th} water quality assessment section; C̅ᵢ̅ is the weighted average concentration of the pollutants of the i^{th} water quantity component, mg/L; and n is a number of the water quantity components.

11. The method for determining the contribution rate of pollution load in the water quality assessment section of the annular river network system based on water quantity constitute according to claim 3, wherein in step iv), a calculation method of the contribution rates of the pollutants of the all water quantity components in the water quality assessment section in the research region is:
$${P}_{i}^{j} = \frac{{ϕ}_{i}^{j} \cdot \overline{{C}_{i}}}{{\sum }_{i = l}^{n} {ϕ}_{i}^{j} \cdot \overline{{C}_{i}}} \qquad ( 2 )$$
wherein Pₜ^{j} is a contribution rate of load of the pollutants of the i^{th} water quantity component in the j^{th} water quality assessment section; ϕₜ^{j} is the water quantity ratio of the i^{th} water quantity component in the j^{th} water quality assessment section; C̅ᵢ̅ is the weighted average concentration of the pollutants of the i^{th} water quantity component, mg/L; and n is a number of the water quantity components.

12. The method for determining the contribution rate of pollution load in the water quality assessment section of the annular river network system based on water quantity constitute according to claim 4, wherein in step iv), a calculation method of the contribution rates of the pollutants of the all water quantity components in the water quality assessment section in the research region is:
$${P}_{i}^{j} = \frac{{ϕ}_{i}^{j} \cdot \overline{{C}_{i}}}{{\sum }_{i = l}^{n} {ϕ}_{i}^{j} \cdot \overline{{C}_{i}}} \qquad ( 2 )$$
wherein Pₜ^{j} is a contribution rate of load of the pollutants of the i^{th} water quantity component in the j^{th} water quality assessment section; ϕₜ^{j} is the water quantity ratio of the i^{th} water quantity component in the j^{th} water quality assessment section; C̅ᵢ̅ is the weighted average concentration of the pollutants of the i^{th} water quantity component, mg/L; and n is a number of the water quantity components.
